(19) United States
(12) Patent Application Publication
(10) Pub. No.: US 2022/0065854 A1
(43) Pub. Date: Mar. 3, 2022

(54) NITROCELLULOSE SHEET COMPRISING IMMOBILIZED IMMUNOGLOBULINS AND LIPID BASED ANTIGENS AND USE THEREOF

(71) Applicants: KEI INTERNATIONAL LIMITED, Hong Kong (HK); TOMORROWS IP LIMITED, Hong Kong (HK)

(72) Inventor: Johannes Theodorus CASTROP, WOUDENBERG (NL)

(21) Appl. No.: 17/312,887

(22) PCT Filed: Dec. 10, 2019
(86) PCT No.: PCT/CN2019/124224

§ 371 Date: Jun. 10, 2021

(30) Foreign Application Priority Data
Dec. 10, 2018 (NL) 2022166

(51) Int. Cl. G01N 33/569; G01N 33/543; G01N 33/92

(52) U.S. Cl. CPC G01N 33/5695; G01N 33/92; G01N 33/54386

(57) ABSTRACT

Disclosed is a nitrocellulose sheet having immobilized antibodies and lipid based antigens. Also disclosed is a kit including the nitrocellulose sheet. and a method using the sheet for detection of antibodies indicative of an infection.

The present invention relates to a nitrocellulose sheet comprising immobilized antibodies and lipid based antigens. The present invention also relates to a kit, comprising said nitrocellulose sheet. The invention also relates to the use of said sheet, in particular in a method for detection of antibodies indicative of an infection.

### INTRODUCTION

Many infectious agents, such as bacteria, viruses and fungi produce antigens which invoke an immune response by the infected subject. This immune response is accompanied by the production of specific antibodies against these antigens. These antibodies are therefore indicative of an infection by an infectious agent.

Many antigens are exposed on or excreted from the outer surface of the infectious agent. A particular important class of antigens are lipid based antigens because many of these are able to invoke a potent immune response.

### SUMMARY OF THE INVENTION

The present invention makes use of the potent immune response invoked by lipid antigens by using them for purposes of diagnosis. In particular, immobilized lipid antigens are used in the invention to detect antibodies in samples derived from subjects suspected of being infected with an infectious agent. This way it becomes possible to diagnose the subject with a particular infection.

Diagnosis of infectious diseases often involves complicated, expensive and time consuming tests and often necessitates specialized laboratories.

Moreover, conventional laboratory tests such as ELISA require large amounts of antigens, which are often hard to purify or synthesize in these amounts. It is therefore desirable to provide a test which requires less antigens.

Furthermore, conventional tests merely diagnose whether a subject is infected with a particular infectious agent or not, but cannot give an indication of the stage of the infection.

The present invention aims to overcome these problems.

In a first aspect the invention relates to a nitrocellulose sheet comprising immobilized thereto at separate positions: immunoglobulin G; immunoglobulin M; and at least one lipid based antigen capable of binding to antibodies produced in response to infection by an infectious agent in a subject.

In a second aspect the invention relates to a kit, comprising the nitrocellulose sheet according to the first aspect of the invention, and further comprising: a container containing an anti-IgG secondary antibody with a conjugated detection label; and a container containing an anti-IgM secondary antibody with a conjugated detection label.

In a third aspect the invention relates to a method for detection of antibodies indicative of an infection comprising: exposing at least one sheet according to the first aspect of the invention to a sample derived from a subject suspected of having an infection; determining the presence of primary antibodies from said sample bound to the antigens immobilized on the sheet by an incubation with a secondary antibody with a conjugated detection label selected from the group comprising anti-IgM secondary antibody or an anti-IgG secondary antibody; and detecting binding of the secondary antibody to said primary antibodies bound to the antigens immobilized on the sheet, wherein binding of said primary antibodies to secondary antibody leads to a positive signal, wherein a positive signal in case of an incubation with an anti-IgM secondary antibody is indicative of an early stage infection and a positive signal in case of an incubation with an anti-IgG secondary antibody is indicative of a later stage infection.

In a fourth aspect the invention also relates to the use of the sheet according to the first aspect for discerning between an early stage infection and a later stage infection and/or for discerning between an active and an inactive infection.

The inventor has found that it is possible to immobilize lipid derived antigens and immunoglobulins onto the same nitrocellulose sheet. The potent immune response to lipid antigens makes it possible to achieve strong detection signals and to obtain reliable results. Importantly, immobilization of the lipid antigens onto the nitrocellulose only requires a limited amount of antigens, which is important because purification and synthesis of antigens is often time consuming and costly. For instance, only 0.2 ng of immobilized antigens is more than sufficient to obtain good signal, while a conventional technique as ELISA requires 250 μg per 96 wells plate. The present invention therefore provides an important reduction of the required amount of antigens.

The sheets are easy to use in a method for detection of antibodies indicative of an infection, without the need for complicated, expensive and time consuming tests and specialized laboratories. In this respect, the kit of the invention comprises parts for performing the method according to the invention without the need for expensive and specialized equipment and the practitioner, such as the researcher would only need standard laboratory equipment. This makes the invention applicable for diagnosis, but also suitable to be used for research purposes not aimed at diagnosis of an infection.

Importantly, the present invention makes it also possible to determine the status of the infection. IgM is the first antibody to appear in response to exposure of an infected subject to an antigen from an infectious agent. The presence of IgM antibodies against antigens of infectious agents therefore indicates an early stage infection with these infectious agents. On the other hand IgG antibodies are generated upon maturation of the antibody response and they participate predominantly in the secondary, later immune response. The presence of IgG antibodies against antigens of infectious agents therefore indicates an later stage infection with these infectious agents. The present invention thus makes it possible to determine whether there is an early stage infection or a later stage infection in an elegant assay using only one type of test substrate, namely a nitrocellulose sheet. This makes it possible to determine immunoglobulin G (IgG) and/or immunoglobulin M (IgM) produced against infectious caused by infectious agents and to determine the status of the infection in one single assay and by using standard laboratory equipment.

### DETAILED DESCRIPTION OF THE INVENTION

The present invention is based on the provision of an easy-to-use test for the presence of antibodies indicative of an infection using a nitrocellulose sheet comprising immobilized thereto at separate positions: immunoglobulin G; immunoglobulin M; and at least one lipid based antigen capable of binding to antibodies produced in response to infection by an infectious agent in a subject.

Exposing the sheet to a sample derived from a subject suspected of having an infection by a particular infectious agent leads to binding to the immobilized antigens if these antigens are derived from or based on antigens of that particular infectious agent. Binding of these antibodies can be detected by using a secondary antibody with a detection label conjugated thereto, such as a reporter enzyme anti-Ig conjugate. Such a conjugate comprises a detection label moiety and a moiety capable of recognizing immunoglobulins (antibodies) bound to the immobilized antigens on the substrate. In other words, these conjugates are capable of recognizing immunoglobulins and are able to generate a detectable signal. In case indeed primary antibodies against the immobilized antigen were present in the sample, a signal will provided by the detection label, such as for instance by a reporter enzyme if the label is a reporter enzyme. A positive signal in case of an incubation with anti-IgM secondary antibody conjugate is indicative of an early stage infection while, on the other hand, a positive signal in case of an incubation with a anti-IgG is indicative of a later stage infection. The IgG and IgM immobilized on the sheet serve as positive/negative controls.

In the context of the invention the antibodies (IgG, IgM, IgA produced in response to infection by an infectious agent in a subject can be considered as primary antibodies, while on the other hand the anti-Ig (antiimmunoglobulin) with conjugated detection label functions as a secondary antibody moiety which can bind to the primary antibody. In the art of immunology primary and secondary antibodies are common expressions for two groups of antibodies that are classified based on whether they bind to antigens or proteins directly (these are primary antibodies) or target another (primary) antibody that in its turn is bound to an antigen or protein (these latter antibodies are secondary antibodies).

While the antibodies produced in response to an infection in a subject are allowed to bind to the immobilized antigens on the nitrocellulose sheet of the invention and as such can be considered as primary antibodies, the anti-Ig conjugates bind to the primary antibodies via their secondary antibody moiety and provide signal detection and amplification via their detection label, such as a reporter enzyme moiety. In the context of the invention, the secondary antibodies can bind to the primary antibodies, which are directly bound to the antigen(s) immobilized on the nitrocellulose substrate. In this respect an anti-IgM secondary antibody is an antibody that specifically recognizes and binds IgM, an anti-IgG secondary antibody is an antibody that specifically recognizes and binds IgG, and an anti-IgA secondary antibody is an antibody that specifically recognizes and binds IgA.

By way of explanation, in an immunolabeling procedure the Fab domain of the primary antibody binds to the immobilized antigen and exposes its Fc domain to the secondary antibody having the detection label. As a result, the Fab domain of the secondary antibody can bind to the Fc domain of the primary antibody. Because the Fc domain is constant within the same animal class (such as for instance within the class of humans), only one type of secondary antibody will be required to bind to many types of primary antibodies within an animal class. This allows the use of only one type of secondary antibody in order to detect multiple types of primary antibodies. Secondary antibodies are therefore commonly commercially available. Moreover, multiple secondary antibodies may bind to a single primary antibody, which increases sensitivity and signal amplification.

The detection label conjugated to the secondary antibody on its turn allows detection of binding. Secondary antibodies can be conjugated to reporter enzymes such as horseradish peroxidase (HRP) or alkaline phosphatase (AP); or fluorescent dyes such as fluorescein isothiocyanate (FITC), rhodamine derivatives, Alexa Fluor dyes, etc.; or other detection labels to be used in various applications, including biotin. In this respect the detection label can be a reporter enzyme such as horseradish peroxidase (HRP) or alkaline phosphatase (AP); or fluorescent dyes such as fluorescein isothiocyanate (FITC), rhodamine derivatives, Alexa Fluor dyes; or other molecules suitable to provide a signal in an immunological assay, as available in the art.

In a preferred embodiment the secondary antibody is a reporter enzyme anti-Ig conjugate. In this case the detection label is a reporter enzyme.

If an incubation with an anti-IgM conjugate as secondary antibody is performed, the IgM immobilized on the sheet serves as a positive control: a positive signal for the binding of the anti-IgM conjugate to the immobilized IgM shows that the test works, while a lesser or absent signal for the binding of the anti-IgM conjugate to the immobilized IgG shows that the signal is specific for binding to IgM.

On the other hand, if an incubation with a anti-IgG conjugate is performed, the IgG immobilized on the sheet serves as a positive control: a positive signal for the binding of the anti-IgG conjugate to the immobilized IgG shows that the test works, while a lesser or absent signal for the binding of the anti-IgG conjugate to the immobilized IgM shows that the signal is specific for binding to IgG.

In case an incubation with a reporter enzyme anti-IgM conjugate is performed, the IgM immobilized on the sheet serves as a positive control: a positive signal for the binding of the anti-IgM conjugate to the immobilized IgM shows that the test works, while a lesser or absent signal for the binding of the anti-IgM conjugate to the immobilized IgG shows that the signal is specific for binding to IgM.

On the other hand, if an incubation with a reporter enzyme anti-IgG conjugate is performed, the IgG immobilized on the sheet serves as a positive control: a positive signal for the binding of the anti-IgG conjugate to the immobilized IgG shows that the test works, while a lesser or absent signal for the binding of the anti-IgG conjugate to the immobilized IgM shows that the signal is specific for binding to IgG.

In one embodiment the nitrocellulose sheet may further comprise immunoglobulin A immobilized thereto. This makes it possible to perform a test determining the presence of antibodies from the sample bound to the antigens immobilized on the sheet by an incubation with a secondary antibody selected from the group comprising anti-IgM conjugate, an anti-IgG conjugate, and an anti-IgA conjugate. In particular these may be selected from the group comprising reporter enzyme anti-IgM conjugate, a reporter enzyme anti-IgG conjugate, and a reporter enzyme anti-IgA conjugate. IgA is the main immunoglobulin found in mucous secretions, including secretions from the genitourinary tract, gastrointestinal tract, prostate and respiratory epithelium, such as sputum. Therefore this embodiment may be particularly suitable to provide an additional possibility to test for infections that affect mucous membranes. For instance, this would in particular be advantageous for testing subjects suspected of tuberculosis infection, because tuberculosis infections lead to high antibody concentrations in the sputum. If in this embodiment an incubation with an anti-IgA conjugate is performed, in this case the IgA immobilized on the sheet serves as a positive control: a positive signal for the binding of the anti-IgA conjugate to the immobilized IgA shows that the test works, while a lesser or absent signal for the binding of the anti-IgA conjugate to the immobilized IgM and IgA shows that the signal is specific for binding to IgA. Apart for the particular advantage for samples based on mucous secretions, testing for IgA antibodies in the sample may also provide a further improvement by confirming results obtained from test on other sheets for IgM and/or IgG antibodies. In case testing for IgA antibodies is envisaged, the kit according to the invention may further comprise a container containing an anti-IgA conjugate as secondary antibody, preferably a reporter enzyme anti-IgA conjugate.

The sample used for purposes of the invention may be any sample which may contain antibodies against antigens of an infectious agent. In one embodiment the sample is a blood derived sample. The sample may be a whole blood sample, a plasma sample or a serum sample. Blood serum is blood plasma without clotting factors and is preferred as plasma. The word plasma in this application may therefore as well refer to (blood) serum. Serum is preferred because it contains less different materials than blood plasma, which may lead to a specific interactions or unwanted biological activity. In addition serum may have a lower viscosity than blood plasma. Using serum therefore may circumvent the need for diluting a sample, which saves time and materials. In another embodiment, the sample may be derived from mucous secretions. In case the sample is a whole blood sample, the sample is may be pre-filtered or separated to plasma or serum if desired. A suitable filter for such a pre-filtering step is a 0.2 micron filter. In principle such a filter step would not be necessary for purposes of the invention.

It may be desirable to dilute the sample prior to incubating it with a sheet according to the invention. If a whole blood sample is not filtered sufficiently or if the patient's physical situation necessitates it, it may be desired to dilute the whole blood sample or plasma or serum. The words plasma or serum in this application may therefore also refer to diluted plasma or serum. It may also be desirable to dilute samples derived from mucous secretions. Dilution may be performed with any suitable diluent, for example a PBS based buffer, such as a blocking buffer. Optionally a detergent may be added in low concentration to the blood/plasma/serum to avoid sticking of components.

The sample may optionally be de-lipidated prior to use, for instance by removing fatty acids, such as cholesterol, if necessary. Cholesterol may lead to incorrect signals because of cross-reactivity.

The subject from which the sample is derived may be a human or another animal. In this respect the invention is suitable for medical and veterinary purposes, and as mentioned above also for research purposes not aimed at diagnosis of an infection. In a suitable embodiment the sample is derived from a human. In this case the secondary antibodies are anti-human Ig conjugates, such as reporter enzyme anti-human Ig conjugates. In this case the secondary antibodies recognize and bind to human immunoglobulin (Ig), for instance human IgG, IgM or IgA.

Said reporter enzyme anti-human conjugates are preferably selected from horseradish peroxidase (HRP) and alkaline phosphatase (AP) conjugates. In this case the step of detecting binding of antibodies in said sample to the immobilized antigens on the sheet involves using reporter enzyme anti-Ig conjugates selected from horseradish peroxidase (HRP) and alkaline phosphatase (AP) conjugates. These conjugates are well known and standard conjugates for visualizing binding of antibodies. These conjugates are commonly used as detection probes because of their simplicity, sensitivity and broad-spectrum applications. Horseradish peroxidase (HRP) and alkaline phosphatase (AP) can be used to detect target proteins through chromogenic, chemiluminescent or fluorescent outputs. These readouts can carried out by any means known to the skilled person.

The lipid based antigens immobilized on the nitrocellulose sheet may be native antigens, or modified antigens which are purified from an infectious agent or synthesized on based on antigens of infectious agents. In this respect it is to be understood that the term “antigen” encompasses any molecule that is capable of specifically binding an antibody, regardless of whether it occurs in nature or whether it is based on such antigen and synthesized.

The antigens immobilized on the nitrocellulose sheet are suitably derived from infectious agents including bacteria, mycoplasma, fungi, parasites, protozoa, viruses, etc. or based on these antigens if synthesized. These infectious agents expose lipid based antigens to the subject's immune system and invoke the production of immunoglobulins.

Well-recognized viruses include Hepatitis A Virus (HAV), Hepatitis B Virus (HBV), Hepatitis C Virus (HCV), Hepatitis D Virus (HDV), Hepatitis G Virus/GB-C Virus (HGV/GBV-C), Human Immunodeficiency Virus types 1 and 2 (HIV-1/2), Human T-cell Lymphotropic Virus types I and II (HTLV-I/II), Cytomegalovirus (CMV), Epstein-Barr Virus (EBV), TT Virus (TTV), Human Herpesvirus type 6 (HHV-6), SEN Virus (SEN-V), and Human Parvovirus (HPV-B19). Bacteria include for instance Treponema Pallidum (TP, the agent of syphilis), Yersinia Enterocolitica, and Staphylococcus and Streptococcus species (common agents of bacterial contamination), Borrelia Burgdorferi (agent of Lyme disease) Pseudomonas spp, Neisseria gonorrhea, Chlamydia, including C. pneumoniae, C. trachomatis, C. psittaci and C. pecorum, Helicobacter, including H. pylori and H. felis, periodontal bacteria including P. gingivalis, and P. sanguis. Mycobacteria, such as Mycobacterium tuberculosis, etc.

Parasites include for instance Plasmodium species (malaria), Trypanosoma Cruzi (Chagas' disease), and Babesia Microti (babesiosis).

Fungi include Candida, yeast-like fungi such as Cryptococcus, Trichophyton, etc.

It is particularly preferred that lipid based are derived from or based on bacterial lipid based antigens because bacteria expose many lipid derived antigens on their cell membrane, for instance the outer membrane. In this respect, the infection may be caused by a bacterial infectious agent.

In a preferred embodiment, the nitrocellulose sheet comprises immobilized thereto a plurality of different lipid based antigens capable of binding to antibodies produced in response to infection by an infectious agent in a subject. This plurality of antigens is preferably derived or based on the same infectious agent. In other words, it is preferred that the lipid based antigens of the plurality of different lipid based antigens are each capable of binding to antibodies produced in response to infection by the same infectious agent. In this embodiment, the use of a plurality of different lipid based antigens, in particular a plurality of different types of lipid based antigens, increases the reliability of a positive test outcome, because the risk of false positive identification of the presence of antibodies invoked by infection is decreased.

Alternatively, the plurality of antigens is derived or based on the different infectious agents. This allows diagnosis of infection with different infectious agents by using only a single kit.

Apart for the difference in occurrence of various types of immunoglobulins during various stages of infection antibodies in the infected subject also the infectious agent may display different antigens in different stages of the infection. It is therefore preferred that of said plurality of different lipid based antigens at least one of said lipid based antigens is capable of binding to antibodies indicative of an early stage infection and at least one other of said lipid based antigens is capable of binding to antibodies indicative of an later stage infection. In increases the reliability of the outcome of the test regarding the state of the infection. Examples of such antigens will be discussed in more detail below.

In a particular preferred embodiment said antigen or antigens is/are derived from or based on mycobacterial lipid based antigen(s). The infection to be assayed may therefore be caused by a Mycobacterium tuberculosis.

Mycobacterium tuberculosis is a pathogenic bacterial species in the family Mycobacteriaceae and the causative agent of most cases of tuberculosis (TB). TB is still one of the leading causes of death in many low and middle income countries. In addition, more and more cases are reported of multi-drug resistant TB. A reliable and fast way of diagnosing tuberculosis such as provided by the present invention is therefore of utmost importance.

In a preferred embodiment, said mycobacterial antigens comprise mycolic acid derived antigens or wherein said antigen is a mycolic acid derived antigen, optionally modified with a functional group to enable immobilisation onto the nitrocellulose sheet.

The mycolic acid derived antigens as referred to in the present application may be derived from mycobacteria selected from virulent and pathogenic mycobacteria. The term mycolic acid derived antigen is to be understood as a compound comprising a mycolic acid residue which is capable of binding to anti-mycolic acid antibodies. Preferably, the mycolic acid antigen is derived from Mycobacterium tuberculosis. Said mycolic acid derived antigen may be at least one selected from the group of mycolic acid, cord factor, chemically modified mycolic acid, chemically modified cord factor, a synthetic mycolic acid derivative, a synthetic cord factor derivative.

The mycolic acid derived antigen may suitably be selected from one or more of mycolic acids obtained from natural sources, synthetically prepared mycolic acids, sulfur-containing mycolic acids, structural analogues of mycolic acids, and mycolic acid wax esters. The mycolic acid derived antigen also includes salts and/or esters of these derivatives.

Natural sources of mycolic acid derivatives include the cell walls of mycobacteria such as Mycobacterium tuberculosis include mixtures of different classes of compounds and different mycolic acid homologues, often as derivatives in which they are bonded to the wall of the cell.

It may be preferred to use synthetically prepared mycolic acids because then it can be exactly determined which and which amount of a particular derivative is used.

Therefore in order to be able to provide a detection with high reliability and reproducible results it is preferred to use semi-synthetic or even more preferred synthetic mycolic acid derivatives which are identical or closely analogous to single compounds found in natural mixtures.

Esters of mycolic acid derivatives can also be used such as esters of alcohols (e.g. monohydric alcohols and polyhydric alcohols) and sugar esters. Sugar esters are particularly preferred. Sugar esters include esters with a monosaccharide, disaccharide or an oligosaccharide. Said saccharides may conveniently include sugar units based on hexoses and/or pentoses. Glucose esters are suitable examples of these esters. Further suitable sugar esters are trehalose esters, including trehalose monomycolates and trehalose dimycolates. For instance cord factors, which are trehalose monomycolates or trehalose dimycolates are well known examples of sugar esters that are suitable. These compounds occur in nature as complex mixtures of different classes of mycolic acids and of different homologues within each class.

Because it is difficult to establish the identity of cord factors present in natural products and to separate individual molecular species it is preferred to use semi-synthetic or more preferably synthetic mycolic acid derivatives for the purposes of the invention. Further, it is known that the structure of the mycolic acid unit affects the biological activity of the cord factor. Suitable semi-synthetic derivatives include semi-synthetic cord-factors which may be prepared by attaching mycolic acids to the sugar group. These semi-synthetic factors however still contain mixtures of different homologue. Therefore particular suitable mycolic acid derivatives for use in the context of the present invention are synthetic cord factors, for example the synthetic cord factors described in WO 2010/08667, i.e. compounds of formula (M)ₓ (S)_{y} (M′)_{z}, wherein x is from 1 to 6, y is from 1 to 12, z is from 0 to 10, each M and each M′ is independently a mycolic acid residue including a β-hydroxy acid moiety and each S is a monosaccharide unit.

Salts of mycolic acid derivatives can also be used, for instance ammonium salts, or alkali metal and alkaline earth metal salts. Sulfur-containing mycolic acids and/or esters or salts thereof may also be used. These compounds are analogues of natural mycolic acid compounds containing sulfur. Mycolic acid wax esters comprise a cyclopropyl group or an alkene and an internal ester group and can be isolated from natural sources or prepared synthetically.

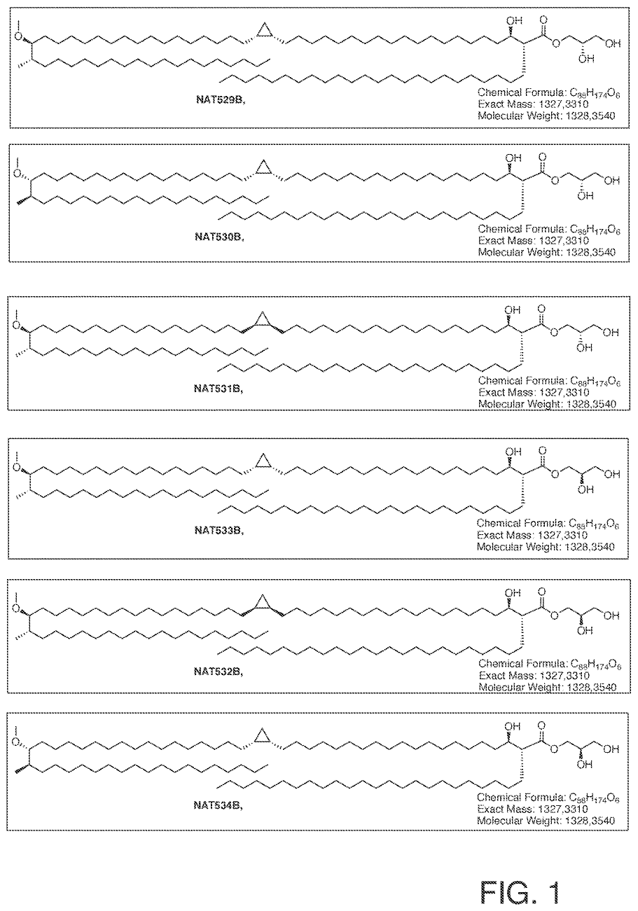

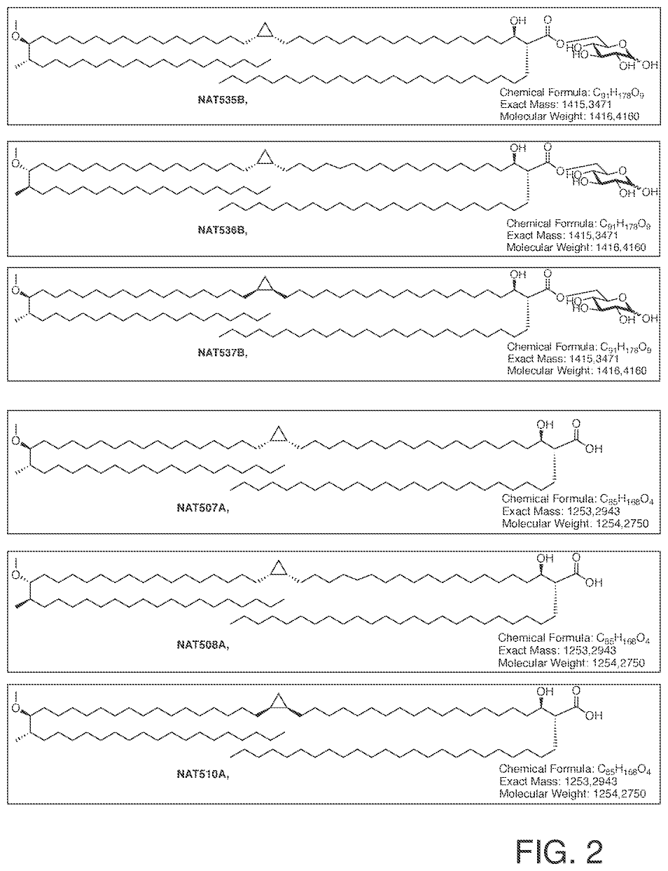

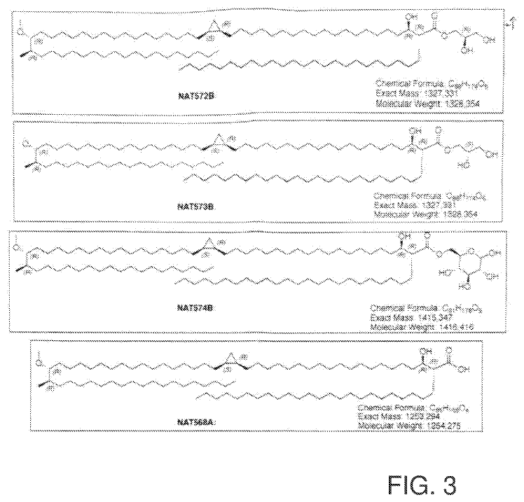

Suitable compounds for use in the detection method of the invention are described inter alia in WO 2016/024116. Suitable examples of synthetic mycolic acids are shown in FIG. 1, FIG. 2 and FIG. 3, optionally to modified with a functional group for immobilization on the nitrocellulose. In this respect this application is also to be understood to describe also the use of one or more of the antigens of FIG. 1, FIG. 2 and FIG. 3 in combination with any substrate other than nitrocellulose for diagnosing tuberculosis.

Another suitable mycobacterial antigen may be a mannosyl phosphoketide antigen. Such a mannosyl phosphoketide antigen may be a compound represented by the following formula (I),

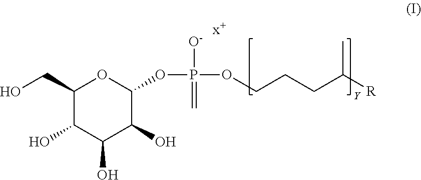

wherein Y is an integer between 1 and 10, X⁺ is a cation or absent, and R is a hydrocarbon group, wherein the antigen is optionally modified with one or more functional groups that enable immobilisation to said nitrocellulose sheet, and enantiomers, diastereoisomers, and mixtures thereof. In case X+ is a cation it is preferred that it is a proton or metal cation, such as a Na or K cation. It is preferred that hydrophobic tail of the mannosyl phosphoketide antigen is rather short. This makes the antigens more soluble in aqueous solutions and thus easier in use. The increased hydrophilicity that results from relatively short hydrocarbon chains makes the nitrocellulose surface to which the antigens are immobilised more hydrophilic. Because of this, interactions of antibodies in the antigen occur easier and the speed of the detection will be enhanced. Moreover, it will be easier to synthesize the antigens in case synthetic antigens are used. In formula (I) Y preferably is an integer between 3 and 9, preferably between 4 and 8, most preferably wherein Y is 5. In formula (I) R preferably is an alkyl group. It is preferred that R is a C1-C15 alkyl, preferably a C5 alkyl or a C7 alkyl, preferably wherein R is an n-C5-alkyl or an n-C7-alkyl. It is preferred that R is an n-C7-alkyl. In formula (I) Y may be modified with one or more functional groups that enable immobilisation to said nitrocellulose sheet. Alternatively, or in addition in formula (I) R may be modified with one or more functional groups that enable immobilisation to said nitrocellulose sheet. Regarding such functional groups, it is preferred that said mannosyl phosphoketide antigens have an alkene or alkyne modification at the end of the hydrophobic tail. It is preferred that said mannosyl phosphoketide antigen is a β-mannosyl phosphomycoketide, optionally modified with one or more functional groups that enable immobilisation to said nitrocellulose sheet. A highly preferred β-mannosyl phosphomycoketide is

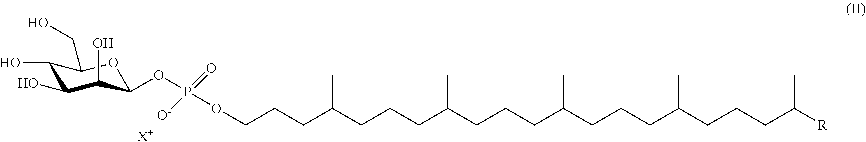

wherein X⁺ is a cation (such as metal cation, e.g. K⁺ or Na⁺ or proton) or absent and R is n-C₇H₁₅ or n-C₅H₁₁, optionally modified with one or more functional groups that enable immobilisation to a nitrocellulose sheet, and enantiomers, diastereoisomers, and mixtures thereof. It is highly preferred that R is n-C₇H₁₅ as this is the form that commonly occurs in Mycobacterium tuberculosis. β-mannosyl phosphomycoketide antigens lead to significant improvement with regard to the sensitivity of detection of markers for tuberculosis. Very high tuberculosis specific binding of antibodies to these antigens is detected in case of samples derived from patients that were tested smear positive. Because β-mannosyl phosphomycoketide molecules exist in Mycobacterium tuberculosis they can be isolated from the bacteria. In this case the natural antigen is used and modified for immobilisation purposes if desired. The levels of β-mannosyl phosphomycoketides in Mycobacteria are rather low, much lower than for instance the levels of mycolic acid. Therefore it is more advantageous to synthesize β-mannosyl phosphomycoketide molecules, either fully chemically or with help of a production organism by means of transgenic expression. This saves considerable costs. It is therefore preferred that the β-mannosyl phosphomycoketide is synthetic. Synthesis can be performed for instance as described in Van Summeren et al., 2006. The above defined mannosyl phosphoketide antigens perform particularly well in case samples derived from smear positive persons are tested, which have an later stage TB infection. Mannosyl phosphoketide antigens are therefore examples of lipid based antigens capable of binding to antibodies indicative of an later stage infection.

Another suitable mycobacterial antigen may be a diacyl glycolipid antigen. The term “diacyl glycolipid antigens” in this application is meant to refer to diacylated trehalose structures as defined in the following structures (III) to (XI). These diacyl glycolipids, which are in fact diacylated trehalose antigens and therefore can also be referred to as diacylated trehalose antigens, or derivatives thereof can be defined as a compound represented by the following formula (III),

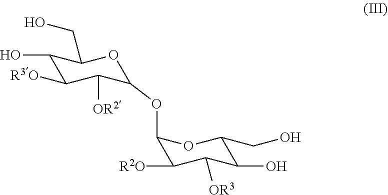

wherein: R² and R³, identical or different, are independently chosen from H, SO³H, SO³⁻ or SO³⁻/M⁺, wherein M⁺ is a cation, preferably a metal cation such as Na⁺ or K⁺; R^{2′} and R^{3′}, identical or different, are acyl groups, wherein the antigen is optionally modified with one or more functional groups that enable immobilisation to a nitrocellulose sheet. The diacyl glycolipids as described herein also include enantiomers, diastereoisomers, and mixtures the compounds of formula (III). In formula (III) R^{2′} and R^{3′}, identical or different, may be

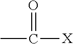

wherein X is independently chosen from an unsaturated or saturated linear or branched hydrocarbon chain, suitably an alkyl group, optionally substituted with one or more substituents and/or modified with one or more functional groups. In a preferred embodiment of formula (III) R² is SO³⁻, SO³H or SO^{3-/}M⁺, wherein M⁺ is a cation and R³ is H. It is preferred that in case R² is SO^{3-/}M⁺ that the cation is Na⁺ or K⁺. In another preferred embodiment of formula (III) R² and R³ are H. It is preferred that in one of R^{2′} and R^{3′} of formula (III) X is a saturated linear hydrocarbon chain optionally substituted with one or more substituents and/or modified with one or more functional groups and wherein in the other of R^{2′} and R^{3′} X is a saturated branched hydrocarbon chain optionally substituted with one or more substituents and/or modified with one or more functional groups. In this respect it is further preferred that in formula (III) one of R^{2′} and R^{3′} is a group represented by the following formula (IV):

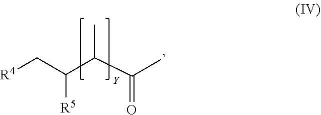

wherein R⁴ is a linear saturated hydrocarbon chain with formula CₙHₙ₊₁, wherein n is an integer between 1 and 20, optionally modified with one or more functional groups, and wherein Y is an integer between 1 and 10, and wherein R⁵ is H or OH, and the other one of R^{2′} and R^{3′} is a linear saturated hydrocarbon chain with formula CₙHₙ₊₁, wherein n is an integer between 1 and 20, optionally modified with one or more functional groups. It is preferred that the acyl chains represented by R^{2′} and R^{3′} are relatively short. This makes the antigens more soluble in aqueous solutions and thus easier in use. The increased hydrophilicity that results from relatively short acyl chains makes the nitrocellulose to which the antigens are immobilised more hydrophilic. Because of this, interactions of antibodies in the antigen occur easier and the speed of the detection will be enhanced. Moreover, it will be easier to synthesize the antigens in case synthetic antigens are used. In this respect in antigens according to formula (III) with short R^{2′} and R^{3′} acyl chains R⁴ may be a linear saturated hydrocarbon chain with formula CₙHₙ₊₁, wherein n is an integer between 1 and 10, such as between 1 and 9, such as between 1 and 8, such as between 1 and 7, such as between 1 and 6 such as between 1 and 5 such as between 1 and 4 such as between 1 and 3, such as 1, 2, 3, 4, 5, 6, 7, or 9, optionally modified with one or more functional groups, and wherein Y is an integer between 1 and 10, such as between 1 and 5, such as between 1 and 4, such as between 1 and 3, such as 1, 2, 3, 4 or 5, optionally modified with one or more functional groups. The other one of R^{2′} and R^{3′} in this respect may be a linear saturated hydrocarbon chain with formula CₙHₙ₊₁, wherein n is an integer between 1 and 15, such as between 1 and 14, such as between 1 and 13, such as between 1 and 12, such as between 1 and 11, such as between 1 and 10, such as between 1 and 9, such as between 1 and 8, such as between 1 and 7, such as between 1 and 6 such as between 1 and 5 such as between 1 and 4 such as between 1 and 3, such as between 1, 2, 3, 4, 5, 6, 7, 8, 9, 10, 11, 12, 13, 14 or 15 optionally modified with one or more functional groups. In case the diacyl glycolipid antigen is present in Mycobacterium, tuberculosis it may be isolated from Mycobacterium tuberculosis. Alternatively, it may be synthesized, for instance by a method as adapted from what is described in EP 1 950 218 A1. If the antigen is not present in Mycobacterium tuberculosis it may be modified from an antigen isolated from Mycobacterium tuberculosis or synthesized. The diacyl glycolipid may suitably be diacylated sulfoglycolipid (Ac₂SGL) as found in Mycobacterium tuberculosis, optionally modified with one or more functional groups. In a preferred embodiment said Ac₂SGL is 2-palmitoyl-3-hydroxyphthioceranoyl-2′-sulfate-α-α′-D-trehalose or 2-stearoyl-3-hydroxyphthioceranoyl-2′-sulfate-α-α′-D-trehalose, optionally modified with one or more functional groups. Ac₂SGL molecules feature a trehalose 2′-sulfate core, and are diacylated with either a palmitic or stearic residue at the 2-position and hydroxyphthioceranic acid, with varying length and methyl substituents, at the 3-position, for instance compound (VI):

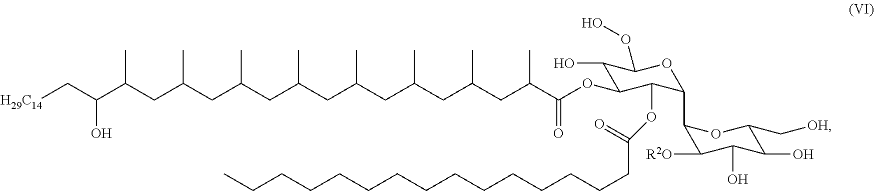

wherein R² is SO³⁻, SO³H or SO^{3-/}M⁺, wherein M⁺ is a kation, preferably a metal cation, preferably Na⁺ or K⁺. Further examples are represented by formulae VII, VIII and IX below.

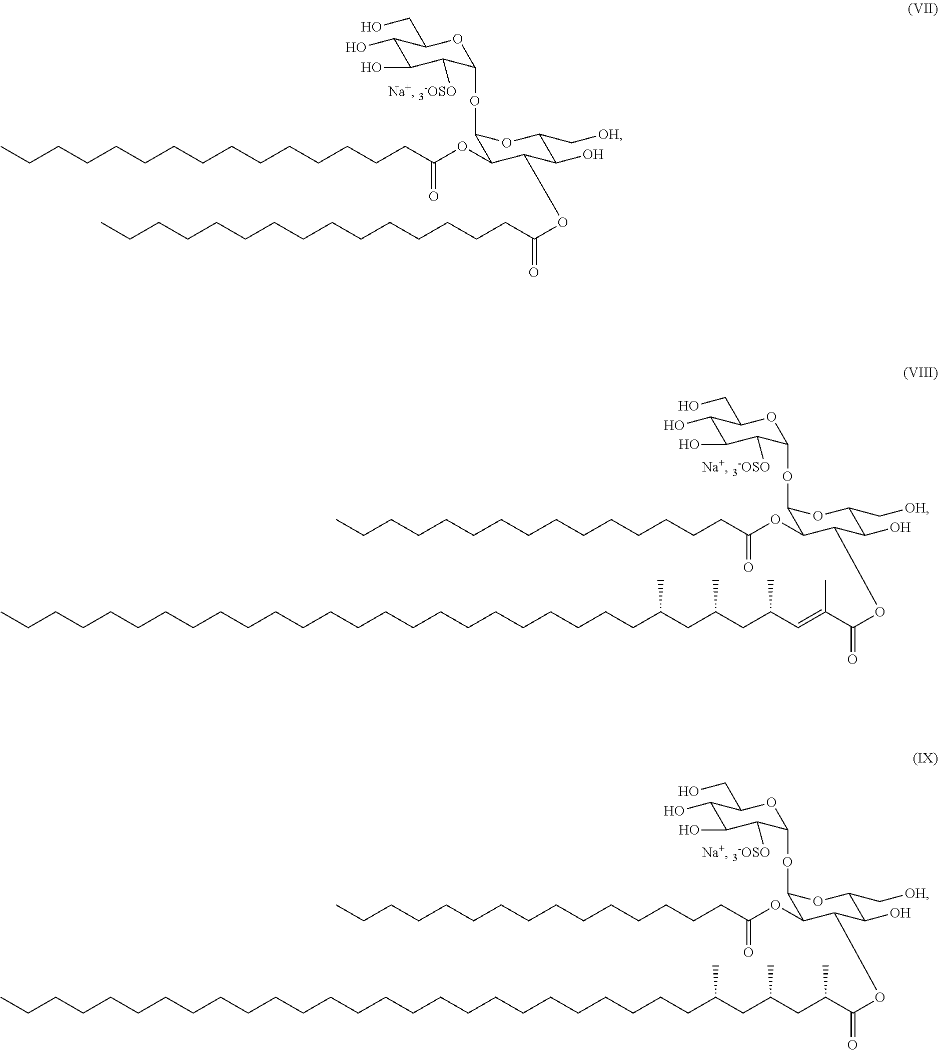

wherein in formulae VII, VIII and IX the SO^{3/}Na⁺ moiety may also be SO³⁻, SO³H and Na+ may also be another cation such as K⁺. In a further embodiment the diacyl glycolipid antigens as defined above may be represented by formula (X):

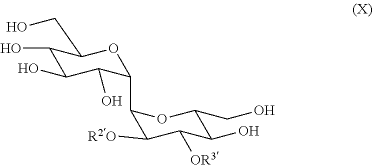

R2′ and R3′ are in formula (X) as defined for formula (III) above. It is preferred that one or both acyl chains herein are modified to enable immobilisation to a nitrocellulose sheet. It is in particular preferred to use a compound as represented by formula (XI):

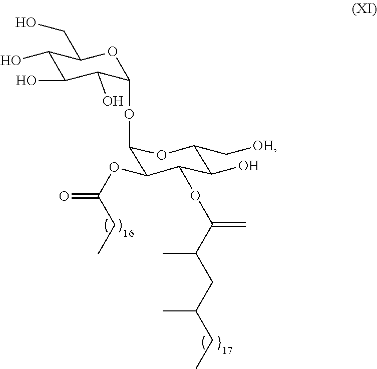

wherein one or both of the acyl chains are modified to enable immobilisation to a nitrocellulose sheet. The acyl chains of the compound of formula (XI) may be substituted for any chain as defined in formula (IV), and the chains may be optionally modified to enable immobilisation to the nitrocellulose.

Suitable modification include the incorporation of a thio group on one of the acyl chains or the incorporation of a unsaturated bond at the end of one of the acyl chains, e.g. a double bond. Further regarding functional groups for immobilization purposes, it is preferred that said diacyl glycolipid antigens have an alkene or alkyne modification at the end of the hydrophobic tail.

Suitable examples of such modified molecules include compounds according to formula (XII), which has an alkene group at the terminus of one of the acyl chains or (XIII) which has a thiol group at the terminus of one of the acyl chains.

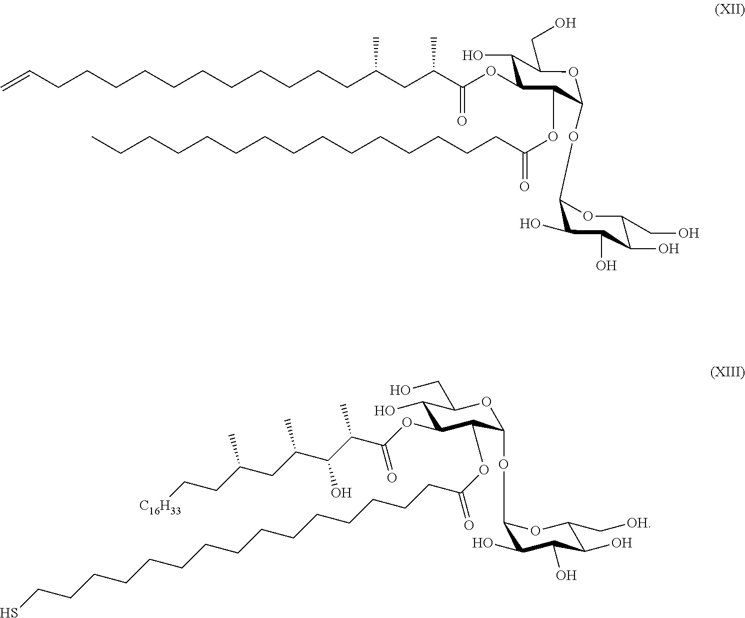

The alkyl chains of the compound of formulae (XII) and (XIII) may be substituted for any chain having the formula CₙHₙ₊₁ or formula (IV) modified with the above shown alkene or thiol group. The diacyl glycolipid antigens provide a means for obtaining high sensitivity of detection of markers for tuberculosis. Very high tuberculosis specific binding of antibodies to these antigens is detected in case of samples derived from patients that were tested smear negative, i.e. which are in an early stage of infection. The above described diacyl glycolipid antigens are therefore examples of lipid based antigens capable of binding to antibodies indicative of an early stage infection.

Further lipid based antigen which can be used as immobilized antigens are tuberculosinyl adenosine antigens. These tuberculosinyl adenosine antigens are compounds represented by the following formula (XIV),

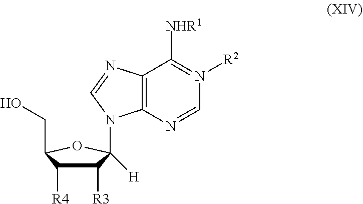

wherein in formula (XIV) R¹ is H or a group with formula (XV)

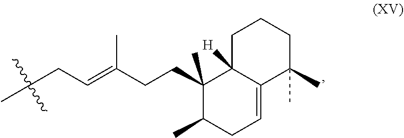

R² is absent or a group with formula (XV), provided that one of R¹ and R² is a group with formula (XV), R³ and R⁴ are selected independently from hydrogen, OH, an acyl chain, a carboxylic acid group comprising an acyl chain, in any combination thereof, wherein the antigens are optionally modified with one or more functional groups that enable immobilisation to a nitrocellulose sheet, and enantiomers, diastereoisomers of the antigens, and mixtures thereof. In this formula it is preferred that R⁴ is OH, and even more preferred that R⁴ and R³ are OH. In formula (XIV) R¹ may be a group of formula (XV) and R² may be absent. In other embodiments R¹ may be H and R² may be a group of formula (XV). In case R² is a group of formula (XV), the nitrogen to which it is attached carries a positive charge. The functional groups that enables immobilisation to a nitrocellulose sheet is preferably included in the group of formula (XV). In case R³ or R⁴ are or comprise an acyl group it is preferred that only one of R³ and R⁴ is or comprises an acyl group, or in particular a fatty acid group. Acyl groups preferably have a suitable length and hydrophobicity to enable immobilisation to a nitrocellulose sheet. The acyl chain may be modified with a functional group to enable immobilisation to a nitrocellulose sheet. An acyl chain may be a mycolic acid chain as described in WO 2014/210327. It is preferred that if the antigen of formula (XIV) has an acyl chain that the acyl chain is rather short. This makes the antigens more soluble in aqueous solutions and thus easier in use. The increased hydrophilicity that results from relatively short hydrocarbon chains makes the detection surface or sensor surface to which the antigens are immobilised more hydrophilic. Because of this, interactions of antibodies in the antigen occur easier and the speed of the detection will be enhanced. Moreover, it will be easier to synthesize the antigens in case synthetic antigens are used. In this respect in antigens with short acyl chains the acyl chain may be a linear or branched C₁-C₂₀ chain, such as a C₅-C₂₀ chain. In a preferred embodiment the antigen of formula (XIV) is represented by a compound of formula (XVI) or (XVII), optionally modified with one or more functional groups that enable immobilisation to a the nitrocellulose sheet. Such a function group may for instance be an azide group, which on its turn can be used in a coupling reaction to couple a biotin group.

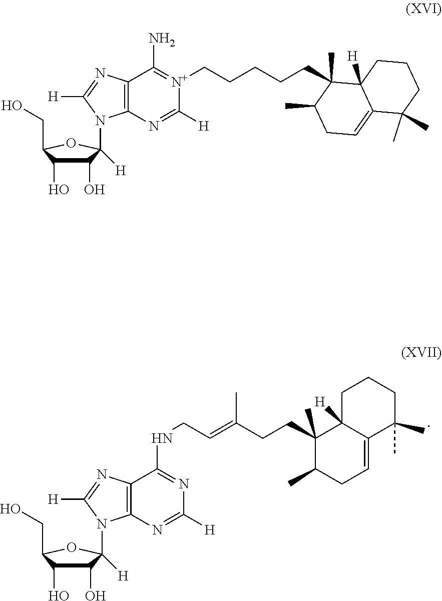

It is preferred that the antigen in accordance with formula (XIV) is selected from the group of 1-tuberculosinyladenosine (as in formula (XVI)), 1-tuberculosinyl-2′-deoxyadenosine, 1-tuberculosinyl-Oacetyladenosine or a combination thereof. These compounds occur naturally in Mycobacterium tuberculosis bacteria and can be isolated therefrom. In this case the natural antigen may be used and modified for immobilisation purposes if desired. The levels of these compounds in Mycobacteria are rather low, much lower than for instance the levels of mycolic acid. Therefore, it is more advantageous to chemically synthesize these molecules, or to synthesize them in a production host micro-organism followed by isolation and optional further purification steps. This saves considerable costs. It is therefore preferred that these compounds are synthetic. Synthetic antigens in accordance with formula (XIV) may for instance be synthetized in accordance with the method described in Buter et al., 2016.

The tuberculosinyl adenosine derived synthetic antigen may also be represented by the following formula (XVII):

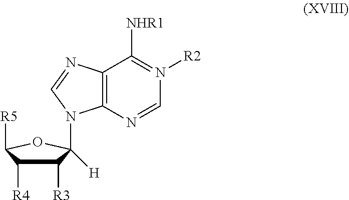

wherein in formula (I) R1 is H or a group with formula (XV)

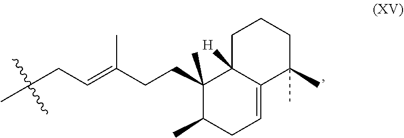

R2 is absent or a group with formula (XV), provided that one of R1 and R2 is a group with formula (XV), R3 and R4 are selected independently from hydrogen, OH or an acyl group, in any combination thereof, and wherein R5 is a linker group, and enantiomers, diastereoisomers of said antigen, wherein said antigen is immobilized to said nitrocellulose sheet via said linker group. In formula (XVIII) R1 may be a group of formula (XV) and R2 may be absent. In other embodiments R1 may be H and R2 may be a group of formula (XV). In case R2 is a group of formula (XV), the nitrogen to which it is attached carries a positive charge. In case R3 or R4 are an acyl group it is preferred that only one of R3 and R4 is an acyl group. For instance R3 or R4 may be an amide, ester, keton or aldehyde or carboxylic acid group. It is possible that R3 is H and R4 is OH. It is also possible that R4 is H and R3 is OH. It is also possible that both R3 and R4 are H. It is preferred that one of R3 and R4 is OH, for instance that R4 is OH or that R3 is OH. It is preferred that R4 is OH and even more preferred that R4 and R3 are OH. Such molecules can be effectively synthesized, for instance in accordance with in accordance with the method described in Buter et al., 2016 and show good antigen presentation. The antigen of formula (XVIII) may suitably be represented by a compound of formula (XIX) or (XX), wherein R5 is a linker group:

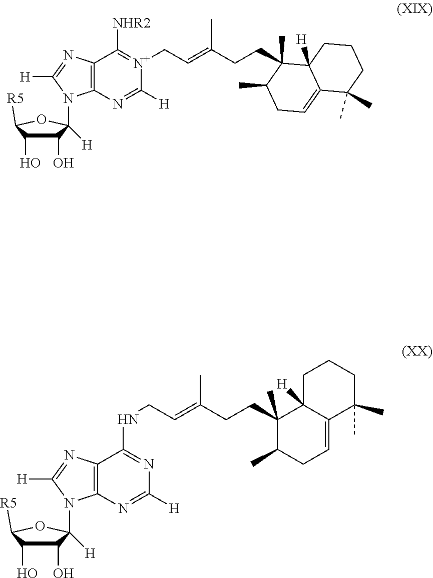

Regarding the linker group of R5 of the antigens of formulae XVIII, XIX and XX, any linker group suitable to immobilize an antigen to a nitrocellulose substrate may qualify and many linker groups suitable for immobilization of antigens are known in the art. Examples of such linkers will be discussed below. In order to enable immobilization to nitrocellulose, the linker preferably contains a functional group that enables immobilization to the substrate. The term “functional group” in this application is therefore to be understood as a group that enables immobilization of the antigen to the nitrocellulose. For this purpose said linker group may comprise a functional group selected from a thio group, an amine group, an aldehyde group, an azide group, a polyhistidine, or a biotin group. A suitable linker for purposes of this invention is a polyethylene glycol (PEG linker) appropriately functionalized to enable immobilization. In a preferred embodiment said linker group is a PEG (polyethylene glycol) containing a functional group that enables immobilization to a nitrocellulose substrate. Many suitable PEG linkers are available and many ways are suitable to couple the antigens to a solid nitrocellulose substrate. The PEG group may for instance be functionalized with group comprises a functional group selected from a thio group, an amine group, an aldehyde group, an azide group, a polyhistidine, or a biotin group. In one embodiment the linker group may therefore be a biotinylated PEG group. In another embodiment said linker group may be a PEG group which contains an azide group, for instance as shown in formula (XXI) below.

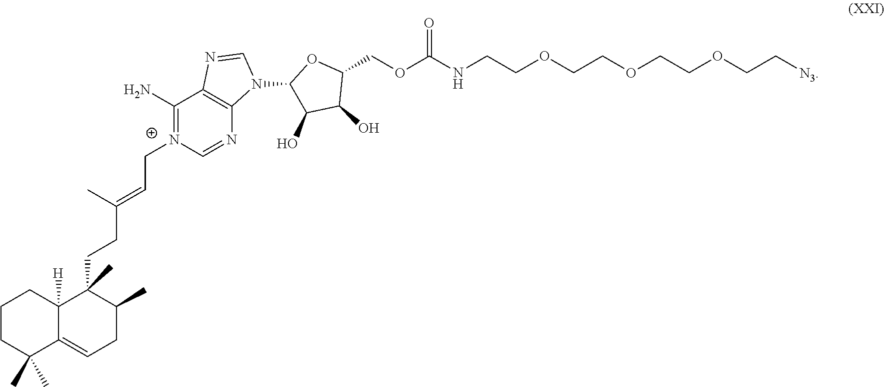

Tuberculosinyl adenosine antigens as defined above are used in a method for detecting a marker for tuberculosis a very high tuberculosis specific binding of antibodies to these antigens is detected. The signal derived from the actual markers for tuberculosis is significantly less obscured by a background signal than when immobilised mycolic antigens are used, so that the signal derived from the actual markers for tuberculosis becomes more pronounced. This way the invention provides a significant improvement with regard to the sensitivity of detection of markers for tuberculosis. The inventor has further found that if tuberculosinyl adenosine antigens as defined above are used in a method for detecting a marker for tuberculosis in combination with other types of antigens that are capable of binding to an antibody which is indicative for the presence of mycobacterial material in a human or animal, a more reliable indication is provided that the subject from which the sample is derived has active tuberculosis. The inventor has now discovered that antibodies against tuberculosinyl adenosine antigens as defined above are not properly detectable in samples derived from subjects vaccinated with a BCG vaccine or from subjects cured from a tuberculosis infection. In other words, if a sample contains a measurable amount of antibodies against tuberculosinyl adenosine antigens as defined above this is an indication of active tuberculosis. This makes it possible to distinguish in a tuberculosis test between subjects with active tuberculosis and subjects wherein the infection is inactive.

In a preferred embodiment the nitrocellulose sheet according to the invention comprises a plurality of different lipid based antigens, which plurality comprises two or more of:

a) at least one mycolic acid derived antigen,
b) at least one tuberculosinyl adenosine antigen
c) at least one diacyl glycolipid antigen
d) at least one mannosyl phosphoketide antigen. Examples of these antigens are described above.

Even more preferably, said plurality of different lipid based antigen comprises:

a) at least one mycolic acid derived antigen
b) at least one tuberculosinyl adenosine antigen
c) at least one diacyl glycolipid antigen; and
d) at least one mannosyl phosphoketide antigen, because as explained above, the presence of antibodies against each of these types of antigens may provide particular information regarding the state or stage of the mycobacterial infection of the subject from which the tested sample is derived. Examples of these antigens are described above.

The nitrocellulose sheet may further comprising one or more protein antigens immobilized thereto, said protein based antigens being capable of binding to antibodies produced in response to infection by an infectious agent in a subject. It is preferred that the lipid based antigen(s) and the protein antigen(s) immobilized to the nitrocellulose are each capable of binding to antibodies produced in response to infection by the same infectious agent. This further improves reliability of the test performed with the nitrocellulose sheet. In case of a bacterial infection such protein antigens may be selected from any known protein antigens of particular infectious agents. For instance in case of the infectious agent is Mycobacterium tuberculosis, possible protein antigens for this purpose may include but are not limited to mycobacterial protein antigens selected from the group comprising: M. Tuberculosis MPT51, MPT63, MPT64, MPT53, M. Tuberculosis 85-A Protein, M. Tuberculosis 85-B Protein, M. Tuberculosis 85-C Protein, M. Tuberculosis CFP-10 Protein, M. Tuberculosis CFP10/ESAT6 Chimera Protein, M. Tuberculosis ESAT-6, Rv3881c, Rv0934 (pstS1), Rv0932c (pstS2) and Rv3006 (LppZ), etc. Such proteins may be recombinant proteins.

Lipid derived antigens and immunoglobulins, and optionally protein derived antigens, may be immobilized first to a nitrocellulose sheet, after which the sheet is cut to sheets or discs. This ensures a equal distribution of the antigens and immunoglobulins. In this respect the nitrocellulose sheet may be in any suitable form, such as a strip or a sheet.

Immobilization to nitrocellulose may take place by any conventional means known in the art. The inventor has observed that lipid derived antigens may be immobilized by means of hydrophobic interaction of one or both of the hydrophobic carbon chains, such as acyl chains, of the antigen with nitrocellulose. It is also possible to modify the antigen with a functional group such as an alkene or alkyne modification at the end of the hydrophobic tail. Mechanisms also include covalent attachment of thiolated antigen to an epoxide-functionalized nitrocellulose membrane, attachment of a biotinylated antigen through a nitrocellulose-binding streptavidin anchor protein, and fusion of an antigen to a novel nitrocellulose-binding anchor protein for direct coupling and covalent attachment through an epoxide thiol linkage using a functionalized nitrocellulose membrane immobilisation.

In accordance with the invention also a kit is provided, comprising the above described nitrocellulose sheet and further comprising: a container containing an anti-IgG secondary antibody with a conjugated detection label; and a container containing an anti-IgM secondary antibody with a conjugated detection label. Optionally the kit further comprise a container containing an anti-IgA secondary antibody with a conjugated detection label.

In a preferred embodiment a kit is provided, comprising the above described nitrocellulose sheet and further comprising: a container containing a reporter enzyme anti-IgG conjugate; and a container containing a reporter enzyme anti-IgM conjugate. Optionally it also contains a reporter enzyme anti-IgA conjugate. The reporter enzymes are for determining the presence of antibodies from said sample bound to the antigens immobilized on the sheet when performing the method of the invention.

Although other reagents, containers and equipment may available in a research lab, a preferred embodiment of the kit may further comprise an incubation tray, configured to receive a number of said sheets, a container with sample diluent; a container with wash buffer; a container with conjugate diluent; and a container with reporter enzyme detection substrate. The abovementioned incubation tray allows testing multiple of said sheets simultaneously. The kit may further be provided with instructions for use.

The following examples are meant to illustrate the principle of the invention and should not be interpreted as limiting the scope of the claims.

### EXAMPLES

### Example 1—An Exemplary Nitrocellulose Sheet

A nitrocellulose strip is provided with immobilized thereto: IgA, IgM and IgG, tuberculosis (TB) lipid antigens including 4 different mycolic acid derived antigens including the natural variant, 1 synthetic tuberculosinyl adenosine antigen; 2 different diacyl glycolipid antigens; and 2 mannosyl phosphoketide antigens, including natural the natural variant. Natural variants are immobilized to the nitrocellulose by the hydrophobic interaction of their hydrophobic tail. The synthetic variants are modified with biotin for improved immobilization to the nitrocellulose. Each antigen is contained on a sheet in an amount of 0.2 ng.

### Example 2: An Exemplary Kit

Table 1 below discloses a kit containing the nitrocellulose strip of example 1.

TABLE 1
contents of an exemplary kit

| Material | Amount | Detail |
|---|---|---|
| Sheets with | 16 | Nitrocellulose strip with controls |
| TB antigens | pieces | and TB lipid antigens |
| Sample | 20 mL | Protein-free sample diluent and |
| diluent | | blocker of non-specific binding |
| Conjugate | 15 μl | Alkaline phosphatase conjugated |
| 4000 × | | monoclonal anti-human |
| concentrated | | IgG or IgM or IgA |
| Conjugate | 20 mL | Protein-free |
| diluent | | conjugate diluent |
| Substrate | 18 mL | NBT/BCIP ready |
| solution | | to use substrate |
| Wash buffer | 160 mL | TBS wash buffer |
| | | with preservative |
| 8 well | 2 | |
| incubation | pieces | |
| tray with cap | | |
| Instructions | 1 | |
| for use | | |

All reagents should preferably be brought to room temperature (20-25° C.) for 1 hour before use. Anti-IgG and anti-IgA are used at 1/2000 dilution (10 μL conjugate in 20 mL conjugate diluent). Anti-IgM is used at 1/4000 dilution (5 μL conjugate in 20 mL conjugate diluent).

Table 2 below lists other materials needed to use the kit, but which are not included in the kit.

TABLE 2
materials which are not included in the kit.

| Material | Number | Detail |
|---|---|---|
| Platform rocker | 1 | Recommended tilt angle |
| | | 8 degrees with 10 rpm |
| Blunt-end forceps | 1 | |
| Pipettes capable | 1 | Preferably an 8 well |
| of delivering 1 mL | | multichannel pipet |
| Pipette capable of | 1 | For pipetting sample |
| delivering 10 μl | | and conjugate |
| and 20 μl | | |
| Vacuum aspirator | | |
| with liquid trap | 1 | Optional but preferred |
| and 0.2 um filter | | |
| Disposable | as | Should fit the 1 mL and |
| pipette tips | required | 20 μl volumes |
| Laboratory timer | 1 | For timing of |
| | | incubation cycles |
| Biosafety cabinet | 1 | For handling |
| | | of potentially |
| | | infectious samples |
| Epson perfection | 1 | |
| v600 scanner or | | |
| equivalent | | |

### Example 3: Use of the Kit

The kit can be used in accordance with the following protocol:
1. Allow all reagents and samples to come to room temperature for 1 hour at 20-25° C.
2. The kit contains a concentrated conjugate which is either anti-IgG, anti-IgM or anti-IgA. Dilute the conjugate by adding 5 (IgM) or 10 (IgG/IgA) μl of the conjugate to the 20 mL vial of conjugate diluent. Alternatively, 2.5 μl may be added to 10 mL of conjugate diluent when analyzing up to 8 samples. Store the undiluted conjugate immediately at 2-8° C. after use. Mark the vial to indicate conjugate has been added. (can also be prepared after step 9)
3. Remove the number of sheets required from the storage tube with forceps.
4. Put the sheets into the 8 well incubation tray so that the black marker line is orientated upwards and on the left of the sheet.
5. Add 1 mL of blocking sample diluent to each of the wells of the incubation tray (The blocker may be for instance a combination of detergent and casein: carbohydrate free, fatty acid free; casein from surmodics; milk blocker from surmodics; Kem and Tec; Soy protein hydrolysate; BSA: fatty acid free, wherein the detergent may be for instance Tween 20, Tween 80, Triton X100; Pluronics 125; Chaps; NP-40)
1. Place the cover on the tray and block the blots in the tray on a rocking platform at 20-25° C. for 30 minutes.
2. Mix the serum or plasma sample with or without delipidation: prior to pipetting by vortexing with the cap on, do not homogenize by pipetting to prevent aerosol formation.
3. Pipet 2.5 μl of sample into the liquid in a single incubation tray well with the sample diluent (400× dilution).
4. Place the cover on the tray and incubate the tray on a rocking platform at 20-25° C. for 30 minutes.
5. Remove the liquid from the trays preferably with a vacuum aspirator or with a pipette.
6. Add 1 mL of wash buffer to the tray and incubate shaking for 30 seconds.
7. Repeat steps 10 and 11 2 times.
8. Remove all liquid from the tray preferably with a vacuum aspirator or with a pipette.
9. Add 1 mL of diluted conjugate to the tray wells.
10. Place the cover on the tray and incubate the tray on a shaking platform at 20-25° C. for 30 minutes.
11. Remove the liquid from the trays preferably with a vacuum aspirator or with a pipette.
12. Add 1 mL of wash buffer to the tray and incubate shaking for 30 seconds.
13. Repeat steps 16 and 17 2 times.
14. Remove all liquid from the tray with a pipette.
15. Add 1 mL of substrate solution to the tray wells.
16. Incubate for 10 minutes at 20-25° C. on a rocking platform.
17. Remove the substrate solution from the trays preferably with a vacuum aspirator or with a pipette.
18. Add 1 mL of wash buffer to the wells.
19. Incubate for 2 minutes on a rocking platform.
20. Remove the liquid from the trays preferably with a vacuum aspirator or with a pipette.
21. Remove the sheets and dry these on a glass surface.
22. After drying apply the sheets onto the printed analysis form. (provide instructions on preparing analysis forms).
23. Scan the sheets using an Epson V600 scanner 600 dpi.
24. Analyze the scan with the analysis software. (provide data analysis instructions).

1. A nitrocellulose sheet comprising immobilized thereto at separate positions:
immunoglobulin G;
immunoglobulin M; and
at least one lipid based antigen capable of binding to antibodies produced in response to infection by an infectious agent in a subject.

2. The nitrocellulose sheet according to claim 1, comprising immobilized thereto a plurality of different lipid based antigens capable of binding to antibodies produced in response to infection by an infectious agent in a subject.

3. The nitrocellulose sheet according to claim 1, wherein the lipid based antigens of the plurality of different lipid based antigens are each capable of binding to antibodies produced in response to infection by the same infectious agent.

4. The nitrocellulose sheet according claim 1, wherein of said plurality of different lipid based antigens at least one of said lipid based antigens is capable of binding to antibodies indicative of an early stage infection and at least one other of said lipid based antigens is capable of binding to antibodies indicative of an later stage infection.

5. The nitrocellulose sheet according to claim 1, wherein said lipid based antigen is derived from or based on a bacterial lipid based antigen or wherein said plurality of different lipid based antigens are derived from or based on bacterial lipid based antigens.

6. The nitrocellulose sheet according to claim 1, wherein said antigen or antigens is/are derived from or based on mycobacterial lipid based antigen(s).

7. The nitrocellulose sheet according to claim 1, wherein said plurality of different lipid based antigens comprise mycolic acid derived antigens or wherein said antigen is a mycolic acid derived antigen.

8. The nitrocellulose sheet according to claim 7, wherein said plurality of different lipid based antigens comprises two or more of:
a) at least one mycolic acid derived antigen;
b) at least one tuberculosinyl adenosine antigen;
c) at least one diacyl glycolipid antigen;
d) at least one mannosyl phosphoketide antigen.

9. The nitrocellulose sheet according to claim 8, wherein said plurality of different lipid based antigens comprises:
a) at least one mycolic acid derived antigen;
b) at least one tuberculosinyl adenosine antigen;
c) at least one diacyl glycolipid antigen; and
d) at least one mannosyl phosphoketide antigen.

10. The nitrocellulose sheet according to claim 1, further comprising one or more protein based antigens immobilized thereto, said protein based antigen(s) being capable of binding to antibodies produced in response to infection by an infectious agent in a subject, preferably wherein the lipid based antigen(s) and the protein based antigen(s) immobilized to the nitrocellulose sheet are each capable of binding to antibodies produced in response to infection by the same infectious agent.

11. The nitrocellulose sheet according to claim 1, further comprising immunoglobulin A immobilized thereto.

12. The kit comprising the nitrocellulose sheet according to claim 1, and further comprising:
a container containing an anti-IgG secondary antibody with a conjugated detection label; and
a container containing an anti-IgM secondary antibody with a conjugated detection label.

13. The kit according to claim 12, further comprising: a container containing an anti-IgA secondary antibody with a conjugated detection label.

14. The kit according to claim 12, wherein said anti-IgG secondary antibody is a reporter enzyme anti-IgG conjugate;
said anti-IgM secondary antibody is a reporter enzyme anti-IgM conjugate; and
said anti-IgA secondary antibody is a reporter enzyme anti-IgA conjugate.

15. The kit according to claim 12, wherein said secondary antibodies are anti-human Ig conjugates.

16. The kit according to claim 14, further comprising:
an incubation tray, configured to receive a number of said sheets,
a container with sample diluent;
a container with wash buffer;
a container with conjugate diluent; and
a container with reporter enzyme detection substrate.

17. The kit according to claim 14, wherein said reporter enzyme anti-human Ig conjugates are selected from horseradish peroxidase (HRP) and alkaline phosphatase (AP) conjugates.

18. A method for detection of antibodies indicative of an infection comprising:
exposing at least one sheet according to claim 1 to a sample derived from a subject suspected of having an infection;
determining the presence of primary antibodies from said sample bound to the antigens immobilized on the sheet by an incubation with a secondary antibody with a conjugated detection label selected from the group comprising anti-IgM secondary antibody and anti-IgG secondary antibody; and detecting binding of the secondary antibody to said primary antibodies bound to the antigens immobilized on the sheet, wherein binding of said primary antibodies to secondary antibody leads to a positive signal, wherein a positive signal in case of an incubation with an anti-IgM secondary antibody is indicative of an early stage infection and a positive signal in case of an incubation with an anti-IgG secondary antibody is indicative of a later stage infection.

19. The method according to claim 18,
wherein the sheet further comprises immunoglobulin A immobilized thereon; and
determining the presence of antibodies from said sample bound to the antigens immobilized on the sheet by an incubation with secondary antibody with a conjugated detection label selected from the group comprising anti-IgM secondary antibody, anti-IgG secondary antibody and anti-IgA secondary antibody.

20. The method according to claim 18, wherein
said anti-IgG secondary antibody is a reporter enzyme anti-IgG conjugate;
said anti-IgM secondary antibody is a reporter enzyme anti-IgM conjugate; and
said anti-IgA secondary antibody is a reporter enzyme anti-IgA conjugate.

21. The method according to claim 18, wherein said secondary antibodies are anti-human Ig conjugates.

22. The method according to claim 20, wherein the step of detecting binding of antibodies in said sample to the immobilized antigens on the sheet involves using reporter enzyme anti-Ig conjugates selected from horseradish peroxidase (HRP) and alkaline phosphatase (AP) conjugates.

23. The method according to claim 18, wherein said infection is caused by a bacterial infectious agent.

24. The method according to claim 23, said infection is caused by a Mycobacterium tuberculosis.

25. The method according to claim 18, wherein said anti-IgG secondary antibody with said conjugated detection label, and said anti-IgM secondary antibody with said conjugated detection label are provided in a kit together with said nitrocellulose sheet.

26. (canceled)

27. The method according to claim 20, wherein said infection is caused by a fungal infectious agent, viral infectious agent or bacterial infectious agent.

28. (canceled)

29. (canceled)
